(19) United States
(12) Patent Application Publication
(10) Pub. No.: US 2023/0096286 A1
(43) Pub. Date: Mar. 30, 2023

(54) MEDICAL INFORMATION PROCESSING APPARATUS, MEDICAL INFORMATION PROCESSING METHOD, AND RECORDING MEDIUM

(71) Applicant: Canon Medical Systems Corporation, Otawara-shi (JP)

(72) Inventors: Yusuke KANO, Nasushiobara (JP); Sho SASAKI, Utsunomiya (JP)

(73) Assignee: Canon Medical Systems Corporation, Otawara-shi (JP)

(21) Appl. No.: 17/935,994

(22) Filed: Sep. 28, 2022

(30) Foreign Application Priority Data
Sep. 30, 2021 (JP) 2021-160739
Sep. 26, 2022 (JP) 2022-152743

(51) Int. Cl. G16H 10/20; G16H 50/70; G16H 50/20; G16H 50/50

(52) U.S. Cl. CPC G16H 10/20; G16H 50/70; G16H 50/20; G16H 50/50

(57) ABSTRACT

A medical information processing apparatus includes processing circuitry. The processing circuitry updates a personal model relating to a target subject. The personal model calculates an effect evaluation value for a medical decision on the target subject. The processing circuitry updates the personal model based on first data corresponding to an effect of the medical decision on the target subject and data limited to a partial range of second data corresponding to an effect of a medical decision on another subject different from the target subject.

### CROSS-REFERENCE TO RELATED APPLICATIONS

This application is based upon and claims the benefit of priority from Japanese Patent Application No. 2021-160739, filed Sep. 30, 2021; and No. 2022-152743, filed Sep. 26, 2022; the entire contents of all of which are incorporated herein by reference.

### FIELD

Embodiments described herein relate generally to a medical information processing apparatus, a medical information processing method, and a recording medium.

### BACKGROUND

Assigning a given medical decision from a number of medical decisions to a patient is practiced in clinical researches and daily medical treatment. Adaptive design, bandit algorithms, etc. are known techniques to adaptively tailor such assignments. In order to maximize the benefit to the patient, it is necessary to deliberately and additionally select (explore), at an appropriate frequency, a medical decision that is less likely to be optimal; however, from the viewpoint of individual patients, there is usually no motivation for such a selection. An exploration may be performed by offering patients a financial incentive; however, this may give rise to a bias on the part of patients who choose to perform such exploration. It is thus difficult to achieve incentive compatibility in the allocation of medical decisions in which the happiness of individual patients is consistent with the happiness of all the patients.

### BRIEF DESCRIPTION OF THE DRAWINGS

FIG. 1 is a diagram showing an exemplary configuration of a medical information processing apparatus according to an embodiment.

FIG. 2 is a flowchart showing medical information processing that is conducted by processing circuitry in accordance with a medical information processing program.

FIG. 3 is a diagram schematically illustrating the medical information processing shown in FIG. 2.

FIG. 4 is a diagram illustrating a range of use according to Working Example 1.

FIG. 5 is a diagram illustrating a range of use according to Working Example 2.

FIG. 6 is a diagram illustrating a range of use according to Working Example 3.

### DETAILED DESCRIPTION

A medical information processing apparatus according to an embodiment includes processing circuitry which updates a personal model relating to a target subject and adapted to calculate an effect evaluation value for a medical decision on the target subject, the processing circuitry updating the personal model based on first data corresponding to an effect of the medical decision on the target subject and data limited to a partial range of second data corresponding to an effect of a medical decision on another subject different from the target subject.

Embodiments of a medical information processing apparatus, a medical information processing method, and a recording medium will be described in detail with reference to the drawings.

In the medical field, efforts to realize clinical decision support (CDS) using technologies based on data and artificial intelligence (AI) are ongoing. AI technologies often use supervised learning, as typified by deep learning, and some technologies are capable of detecting lesions with greater accuracy than humans, especially in the diagnostic imaging domain.

However, the following circumstances are expected to hinder the realization of clinical decision support (CDS) through supervised learning. First, improvement in accuracy requires an enormous amount of data, which would incur large costs for data collection. Second, despite the related high accuracy, a proper clinical decision may not result.

The second issue is technically synonymous with a problem that supervised learning, while being capable of revealing a correlation, cannot demonstrate a causal sequence. For example, even if an AI model can predict the within-5-year death of a heart failure patient with 100% accuracy, such an AI model cannot assist a doctor who wants to know how to extend the remaining life of such patient to 5 years or longer.

Demonstrating a causal sequence in medical activities to thereby specify the optimal medical option is yet to be realized by the current form of supervised learning, which falls under the role of randomized controlled trials (RCT) (medical statistics). In other words, as long as the current AI technologies prevail, a clinical decision support (CDS) for the optimized treatment cannot be realized in the true sense, and randomized controlled trials (RCT) will continue to be used.

Randomized controlled trials (RCT) also involve many problems. For example, these problems include, first, the significant amount of time required to complete the research, which makes it impossible to follow the latest trends, thus the resultant findings could easily become obsolete. Second, the research is very costly, which is also accompanied by a risk of failure in proving the hypothesis, thus efficiency is not guaranteed. Third, in general, eligibility criteria set for the research are strict, which could render the resultant findings inapplicable for ordinary patients (i.e., result in low external validity). Fourth, as a control, only an average intervention effect can be employed, which does not allow for effects that vary among individual patients to be specified. This, for example, precludes the possibility of providing personalized medicine. Fifth, statistical significance could be overly relied upon, which may easily lead to publication bias, p-hacking, etc.

In particular, currently, treatment methodologies are frequently updated and renewed, which further underscores the importance of personalized medicine. Thus, the limits of randomized controlled trials (RCT) are becoming apparent.

Accordingly, there is a demand for efficient optimization of medical decisions that does not require a large amount of data in advance and that can be performed without randomized controlled trials (RCT). Furthermore, for this purpose, formulation of models for clinical decision support (CDS) is desired.

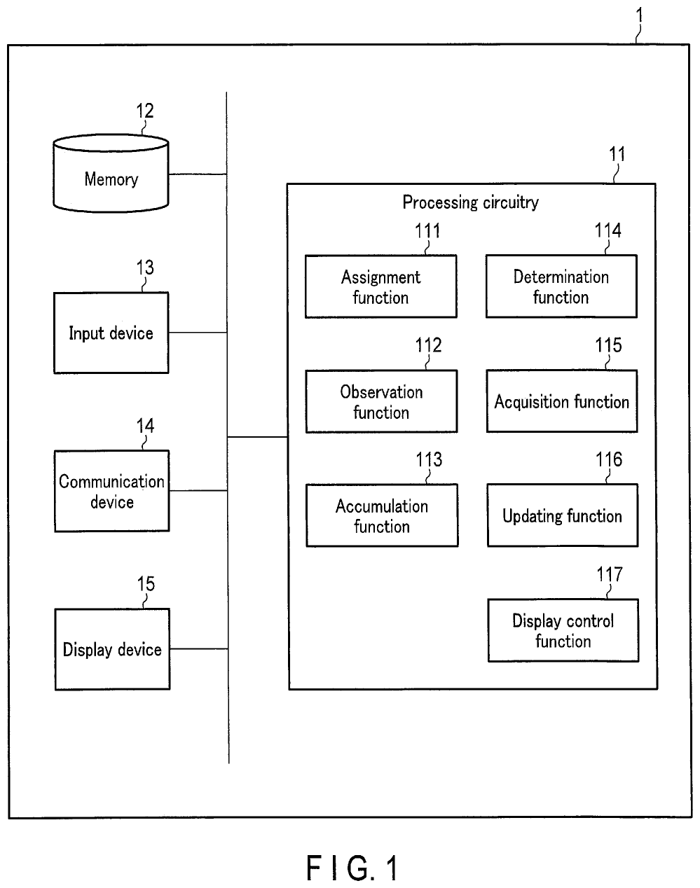

FIG. 1 is a diagram showing an exemplary configuration of a medical information processing apparatus 1 according to an embodiment. As shown in FIG. 1, the medical information processing apparatus 1 is an information processing terminal which may be a computer, etc., including processing circuitry 11, a memory 12, an input device 13, a communication device 14, and a display device 15. The processing circuitry 11, the memory 12, the input device 13, the communication device 14, and the display device 15 are connected with one another via a bus so that they can mutually input and output signals.

The processing circuitry 11 includes one or more processors such as a central processing unit (CPU), a graphics processing unit (GPU), and so on. By running one or more medical information processing programs, the processing circuitry 11 implements various functions, including an assignment function 111, an observation function 112, an accumulation function 113, a determination function 114, an acquisition function 115, an updating function 116, and a display control function 117. Note that the functions 111 to 117 are not limited to the implementation through a single processing circuitry component. Multiple independent processors may be employed together to form processing circuitry so that the respective processors run the programs to realize the functions 111 to 117. Moreover, the functions 111 to 117 may be respective programs serving as modules to constitute a medical information processing program. Such programs may be stored in the memory 12.

The memory 12 is a storage device adapted to store various types of information sets, such as one or any combination of a read only memory (ROM), a random access memory (RAM), a hard disk drive (HDD), a solid state drive (SSD), an integrated circuit memory device, and so on.

Other than such storage devices, the memory 12 may be one or any combination of non-transitory computer-readable recording media such as a compact disc (CD), a digital versatile disc (DVD), and a flash memory, or a driver that reads and writes various types of information sets with semiconductor memory devices. Also, the memory 12 may be located within an external computer connected via a network.

The input device 13 receives various input operations from an operator and converts the received input operations into electrical signals for output to the processing circuitry 11. More specifically, the input device 13 is coupled to one or more input instruments such as a mouse, a keyboard, a track ball, switches, buttons, a joystick, a touch pad, and a touch panel display. The input device 13 outputs electric signals corresponding to input operations received by such input instruments to the processing circuitry 11. Note that the input device 13 may be provided on an external computer connected via a network, etc.

The communication device 14 is an interface for exchanging various types of information sets with one or more external computers. The communication device 14 performs data communication according to the standard suitable for medical information communication, such as a digital imaging and communications in medicine (DICOM).

The display device 15 displays various types of information sets according to the display control function 117 of the processing circuitry 11. The display device 15, for example, may be a liquid crystal display (LCD), a cathode ray tube (CRT) display, an organic electroluminescence display (OELD), a plasma display, or any other display available. The display device 15 may instead be a projector.

The processing circuitry 11 according to the embodiment, with the assignment function 111, determines a medical decision to be assigned to a testee such as a patient, based on an effect evaluation value for the medical decision. Such effect evaluation values for medical decisions are calculated using personal models adapted to calculate the effect evaluation values for medical decisions. The testee receives a medical act corresponding to the assigned medical decision. An effect attributable to the medical act is produced for the testee. The processing circuitry 11, with the observation function 112, observes the effect produced for the testee. The effect is represented by a numerical value. This numerical value will be called an “effect observation value”. With the accumulation function 113, the processing circuitry 11 accumulates effect observation values in the database.

With the updating function 116, the processing circuitry 11 updates a personal model relating to the target subject and adapted to calculate an effect evaluation value for a medical decision based on first observation data corresponding to the effect of the medical decision on the target subject and observation data limited to a partial range of second observation data corresponding to the effect of the medical decision on another subject different from the target subject. The target subject is a testee for which the personal model is trained. The target subject and another subject may be defined as the testee himself/herself, a group of testees including at least one testee, a hospital, or a hospital group including at least one hospital. The first observation data is series data including at least multiple effect observation values respectively corresponding to multiple observation times of the effect of the medical decision on the target subject. The second observation data is series data including multiple effect observation values respectively corresponding to multiple observation times of the effect of the medical decision on another subject. The medical act and other feature amounts may be associated with the effect observation value of each observation time. The first observation data and the second observation data are accumulated in a database. With the determination function 114, the processing circuitry 11 determines the partial range. With the acquisition function 115, the processing circuitry 11 acquires data limited to a partial range of the second observation data. With the display control function 117, the processing circuitry 11 presents various data sets through the display device 15.

The testee, in the context of the present embodiment, is typically a single patient suffering from a disease. However, the testee assumed by the embodiment is not limited to this but may be a patient group constituted by two or more patients. Also, the testee in the embodiment is not limited to a person suffering from a disease, but may be a healthy person. The models may be employed for respective doctors, respective treatment departments, respective hospitals, or respective geographical areas, and may also be employed for respective testees.

Each model according to the embodiment is a function for calculating the effect evaluation value for a medical decision. This function may be a function defined by a manual operation, a function defined by an experimental approach or a deterministic approach, a function formed through training in machine learning, or a function defined by another method. The effect evaluation value is an index value for evaluating the effect of an assignment candidate medical decision, and examples of such an index value include a confidence interval of the effect, an expected value of the effect, the highest value among such expected values, a difference from the highest expected value, and so on. The term “personal model” according to the present embodiment refers to a model specifically adapted for the target subject.

The embodiment assumes types of medical decisions including execution/non-execution-related decisions, content-related decisions, amount-related decisions, and time-related decisions. Examples of the execution/non-execution-related decisions include a decision as to whether or not to execute a surgery or whether or not to execute a blood test. Examples of the content-related decisions include a decision for the selection of a drug to be used or the selection of a disease name to which diagnosis is conducted. Examples of the amount-related decisions include a decision for the selection of a dose of pharmaceuticals or the selection of a rehabilitation period. Examples of the time-related decisions include a decision for the selection of a time to visit a hospital or the selection of a time to execute a surgery.

Persons who make medical decisions are not limited to medical professionals such as doctors, nurses, and other health-care providers, but may include testees themselves, family members of testees, and any other persons associated with testees. Medical decisions are not always required to be advanced or sophisticated decisions for healthcare, nor are they limited to the purposes of health improvement. That is, medical decisions may cover decisions that lead to a worse result for medicine, health, etc. For example, a decision as to whether or not a healthy individual smokes a cigarette at a given timing is also assumed to be a medical decision.

A medical decision consequently results in a certain effect. Such an effect is called a “reward” or an “outcome”. Effects discussed in the context of the embodiment are assumed to be, for example, clinical outcomes, patient-reported outcomes, and economic outcomes. Examples of the clinical outcomes include a morbidity rate (including whether or not a subject suffers from a disease or the like), a 5-year survival rate (including whether or not a subject is alive), a complication incidence rate (including whether or not a subject suffers from a complication), a re-hospitalization rate (including whether or not a subject is hospitalized again), test values or an improvement degree of test values, and a level of independence in daily life. Examples of the patient-reported outcomes include a subjective symptom, a subjective health condition, a treatment satisfaction level, and a subjective happiness level. Examples of the economic outcomes include medical costs, invested medical resources, and a length of stay at a hospital.

The effects take the form of numerical representations, on which a superiority-and-inferiority assessment can be done for learning. Numerical values may be assigned to items that are not originally numerical representations. The effects may include those readily observable after a medical decision. For example, an effect of a medical decision as to whether or not to send a message prompting an exercise to the smartphone of a healthy individual may be a fact of conducting or not conducting an exercise within 5 minutes of the receipt of the message.

The effects may take into consideration the costs required for a medical decision. For example, in the instances of a smartphone message as discussed, sending a message would provide merits of more easily obtaining the effects than not sending a message, but it could concurrently cause demerits of incurring a communication cost, restricting the user activities, etc. One exemplary implementation for taking such merits and demerits into consideration may be to assume, for example, that sending a first message always incurs a cost of “5” and conducting an exercise gives a reward of “100”, and calculating the value of the effect as 100 (reward)−5 (cost)=95. In this manner, effects reflecting cost effectiveness can be obtained.

Feature amounts according to the embodiment include one or more attributes and/or one or more conditions of a testee. The attributes here are information items which, by nature, do not change in relation to a medical decision just performed, and they include the sex, age, etc. of a testee. The conditions here are information items which, by nature, do change in relation to a medical decision just performed, and they include a current blood pressure, blood pressure value, a current blood glucose level, etc. of a testee.

A description will be given of an exemplary operation of the medical information processing apparatus 1 according to the embodiment.

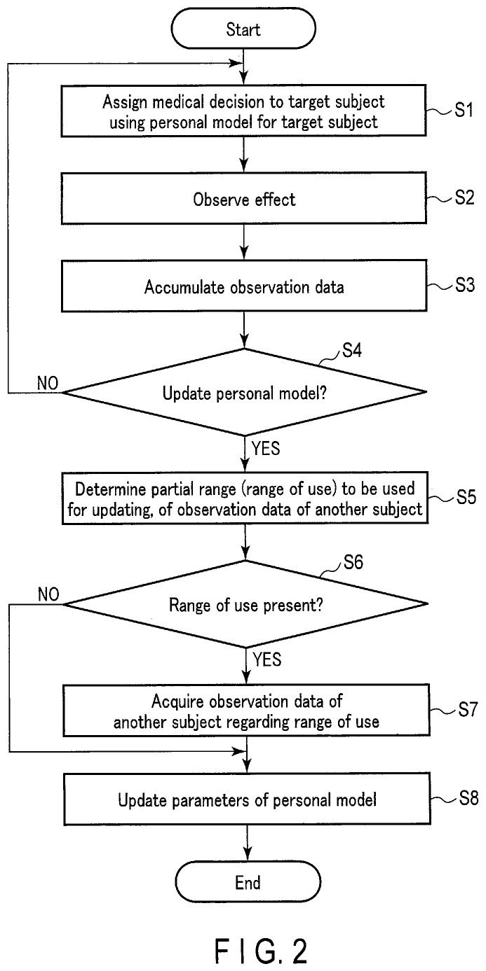

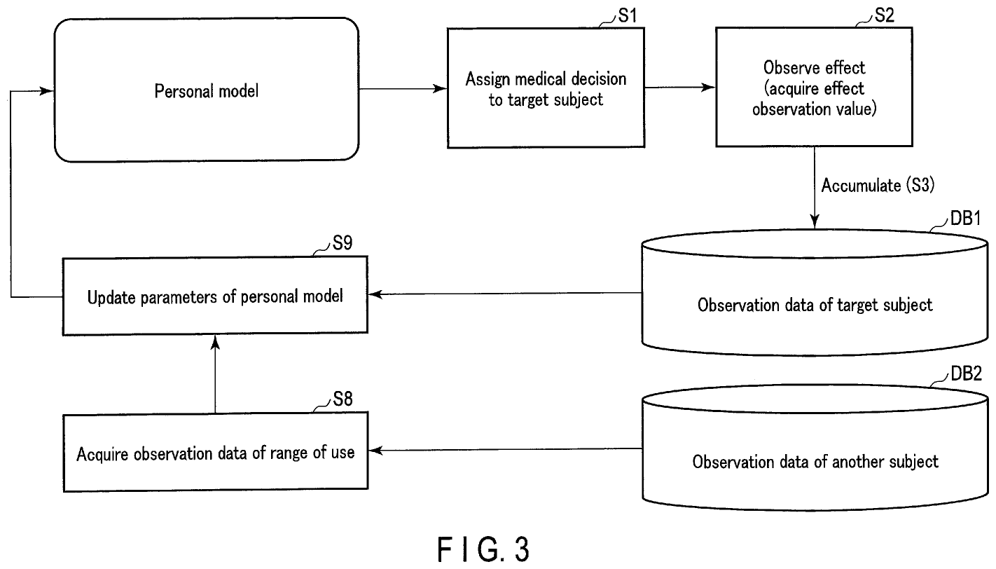

FIG. 2 is a flowchart showing medical information processing conducted by the processing circuitry 11 in accordance with a medical information processing program. FIG. 3 is a diagram schematically illustrating the medical information processing shown in FIG. 2.

As shown in FIGS. 2 and 3, with the assignment function 111, the processing circuitry 11 assigns a medical decision to a target subject using a personal model for the target subject (step S1). More specifically, the assignment is performed through the following processes.

The processing circuitry 11 includes one or more personal models for calculating effect evaluation values for medical decisions, specifically adapted for the target subject. The personal models include multiple structure models for handling multiple medical decisions, respectively. Each structure model is a model for calculating effect evaluation values for the corresponding medical decisions.

Basically, the processing circuitry 11 calculates an effect evaluation value by applying, to a personal model, observation data of the target subject obtained from a previous or earlier observation prior to the processing time (processing step). The effect evaluation value may be calculated based solely on observation data that is acquired from an observation conducted immediately before the processing time, or based on observation data acquired through multiple observations conducted prior to the processing time.

The processing circuitry 11 assigns an appropriate medical decision from among multiple medical decisions based on multiple effect evaluation values and according to the technique utilizing adaptive design or bandit algorithms. There are no particular restrictions on actual algorithms for use as the bandit algorithms. In the disclosure of the embodiment, the bandit algorithms are algorithms for solving a problem of sequentially selecting appropriate medical decisions from multiple medical decisions (options) so as to maximize the sum of the effects. The bandit algorithms in the embodiment include not only narrowly-defined bandit algorithms where effects do not depend on a feature amount, but also contextual bandit algorithms where effects depend on a feature amount. The bandit algorithms in the embodiment further include reinforcement learning for solving a sequential decision-making problem where conditions vary according to the medical decisions to date. Examples of the actual algorithms that may be used as the bandit algorithms in the embodiment include epsilon greedy, Thompson sampling, linear Thompson sampling, posterior sampling for reinforcement learning (PSRL), and Bayesian deep Q-networks (BDQN).

The number of structure models included in the personal model according to the present working example is set according to the number of medical decisions from which the selection is made. The number of structure models is not particularly limited, and may be any number that is equal to or greater than two. However, in explaining the working examples set forth below, it is assumed that the number of structure models is two; namely, the structure model corresponding to the medical decision A and the structure model corresponding to the medical decision B. In this case, the processing circuitry 11 determines an appropriate one of the two medical decisions A and B as an assignment target.

In the present working example, it is assumed, as an example, that the parameter or parameters of the personal model is/are updated by Thompson sampling, which is a type of bandit algorithm. Thompson sampling is a technique in which expected value parameters of an effect are modeled in a Bayesian statistical framework, and to which a policy based on the probability matching method is applied. Bayesian statistics is a statistical theory in which the probability is interpreted as being variable whenever new information is obtained, and a probability (or a probability distribution) before and after the obtainment of the information is updated based on Bayes' theorem. The probability matching method is a method of selecting an option with “a probability that the option is the maximum expected value” at each time, and is a technique (probabilistic policy) in which the adopted option is subject to randomization. In the probability matching method, the “probability in which the option is the maximum expected value” can be formulated by a given method, and a method of calculating such a probability through a Bayesian approach is Thompson sampling.

An update of a probability distribution in Thompson sampling is calculated by assuming a conjugate prior distribution. It is assumed that a probability distribution of an effect produced on a testee as a result of the medical decision follows the Bernoulli distribution. In this case, the beta distribution, which is the conjugate prior distribution for the Bernoulli distribution, is used as the posterior distribution of the expected value of the effect of the medical decision. The Bernoulli distribution is a discrete probability distribution in which “1” is taken with a probability p and “0” is taken with a probability 1−p. The posterior distribution of the expected value of the effect of the medical decision A is expressed by Beta (α_{A}, β_{A}), using parameters α_{A} and β_{A}. The parameter α_{A} is the number of observations of the effect observation value “1” as the effect of the medical decision A, and the parameter α_{B} is the number of observations of the effect observation value “0”. Similarly, the posterior distribution of the expected value of the effect of the medical decision B is expressed by Beta (α_{B}, β_{B}), using the parameters α_{B} and β_{B}.

The effect evaluation value Y^{A} is calculated in accordance with the following formula (1), based on the Bernoulli distribution which the effect of the medical decision A follows and the parameter p_{A} which defines the Bernoulli distribution. Similarly, the effect evaluation value Y^{B} is calculated in accordance with the following formula (2), based on the Bernoulli distribution which the effect of the medical decision B follows, and the parameter p_{B} which defines the Bernoulli distribution. The formula (1) is a mathematical expression of a structure model relating to the medical decision A, and the formula (2) is a mathematical expression of a structure model relating to the medical decision B.

Y^{A}=Bern(p_{A}) (1)

Y^{B}=Bern(p_{B}) (2)

In Thompson sampling, the processing circuitry 11 sequentially determines the current option (the medical decision A or the medical decision B) based on the selection of a series of medical decisions obtained from an observation conducted prior to the processing time and the effect observation value. More specifically, the processing circuitry 11 randomly generates the expected values μ_{A} and μ_{B} of the effects of the respective options A and B from the posterior distributions Beta (α_{A}, β_{A}) and Beta (α_{B}, β_{B}), selects a medical decision corresponding to the maximum expected value from the expected values μ_{Z} and μ_{B}, and assigns the selected medical decision to the testee.

A medical act corresponding to the medical decision of the assignment target is conducted by a medical professional, etc. on the target subject, and an effect is produced for the target subject. After step S1, with the observation function 112, the processing circuitry 11 observes the effect produced on the target subject (step S2).

The effect is observed in the form of a numerical value or values, i.e., effect observation values. For example, if the assigned medical decision indicates “Execute a surgery”, information such as a 5-year survival rate, the presence of complication incidence, and so on are acquired as the effect observation values. The effect observation values may be acquired by any method. For example, an operator may input them via the input device 13, or a testing instrument may input its measurement values. The effect observation values may be received from one or more external computers via the communication device 14.

After step S2, the processing circuitry 11 accumulates observation data including an effect observation value in a database DB1 (step S3). The database DB1 is a computer including a memory that accumulates the observation data of the target subject. The database DB1 is connected to the medical information processing apparatus 1 via the communication device 14 so that data transmissions and receptions are enabled among them. It is assumed that the observation data includes at least an identifier of the testee and the effect observation value. The observation data may further include a given feature amount such as the sex of the testee.

After step S3, with the updating function 116, the processing circuitry 11 determines whether or not to update the personal model (step S4). The timing of updating the personal model is not particularly limited, and may be based on the number of samples of observation data accumulated in the database DB1, or based on a period of time that has passed since a reference time. The timing based on the number of samples of observation data accumulated in the database DB1 may be set to, for example, the timing when a given number of (e.g., one, three, or ten) samples of observation data are accumulated in the database DB1. The timing based on the period of time that has passed since the reference time may be set to, for example, a timing when a freely set period of time (e.g., one week, two weeks, one month, etc.) has passed since the previous update time of the personal model or the time of start of use of the personal model.

If it is determined in step S4 that the personal model is not to be updated (step S4: NO), the processing circuitry 11 returns to step S1, and repeats steps S1 to S4 until it is determined that the personal model is to be updated.

If it is determined in step S4 that the personal model is to be updated (step S4: YES), the processing circuitry 11 determines, with the determination function 114, a partial range (hereinafter referred to as a “range of use”) to be used for the updating, of the observation data of another subject (step S5). In step S5, the processing circuitry 11 determines whether or not the range of use exists, namely, whether or not to use observation data of another subject. If it is determined that the range of use exists, the processing circuitry 11 determines a specific range of the range of use. More specifically, the processing circuitry 11 determines the range of use based on an activity and/or a feature amount of the target subject. The presence or absence of the range of use and the determination method can be determined by a variety of methods. The presence or absence of the range of use and the determination method of such a range will be described in several different working examples. Said another subject is freely set from among one or more testees different from the target subject. The number of said another subjects may be one or more than one.

If the range of use is determined in step S5, the processing circuitry 11 determines, with the acquisition function 115, the presence or absence of the range of use (step S6). If it is determined in step S6 that the range of use is absent (step S6: NO), the processing proceeds to step S8.

If it is determined in step S6 that the range of use is present (step S6: YES), the processing circuitry 11 acquires, with the acquisition function 115, observation data of another subject regarding the range of use (step S7). The observation data of another subject is accumulated in a database DB2 in a method similar to that of the target subject. The database DB2 is a computer including a memory that accumulates observation data of another subject. The database DB2 is connected to the medical information processing apparatus 1 via the communication device 14 so that data transmissions and receptions are enabled among them. The database DB2 and the database DB1 may be either the same computers or different computers. To acquire observation data of another subject regarding the range of use, any one of the following two aspects may be adopted. First Aspect: The processing circuitry 11 acquires an entire range of observation data relating to another subject from the database DB2, and then extracts observation data relating to a range of use from the acquired entire range of observation data. Second Aspect: The processing circuitry 11 acquires observation data of the range of use relating to another subject from the database DB2.

If it is determined in step S6 that the range of use is absent (step S6: NO) or step S7 is performed, the processing circuitry 11 updates, with the updating function 116, parameters of the personal model (step S8). In step S8, the processing circuitry 11 updates the parameters of the personal model based on the observation data of the target subject and the observation data of another subject included in the range of use determined based on the activity and/or the feature amount of the target subject. If, for example, an effect observation value R^{A}-relating to the target subject has been acquired, parameters α_{A} and β_{A} are updated based on observation data including the effect observation value R^{A}. The parameters α_{A} and β_{A} respectively refer to the number of times when “1” and “0” are observed, and are therefore updated in accordance with the formula (3) below. That is, the processing circuitry 11 increments “1” to the parameter α_{A} if the effect observation value R^{A} is “1”, and increments “1” to the parameter β_{A} if the effect observation value R^{A} is other than “1”.

if Y^{A}=1 then α_{A}←α_{A}+1 else β_{A}←β_{A}+1 (3)

In the present embodiment, it is assumed that the personal model differs according to the testee. Accordingly, the parameters α_{A} and β_{A} take a value that differs according to the testee. Assuming that the parameters of the personal model of the testee X are α_{A}^{x} and β_{A}^{Y}, α_{A}^{X} and β_{A}^{Y} are updated based on the effect observation value R_{A}^{X} as a result of selection of the medical decision A for the patient X. Whether or not the update is performed based on the effect observation value R_{A}^{Y} observed for another testee Y is appropriately controlled.

If it is determined in step S6 that the range of use is absent (step S6: NO), the processing circuitry 11 updates parameters of the personal model based on only the observation data of the target subject (step S8). Specifically, the parameters α_{A}^{X} and β_{A}^{Y} of the personal model for the testee X are updated based on the effect observation value R_{A}^{X} relating to the target subject.

If it is determined in step S6 that the range of use is present (step S6: YES), the processing circuitry 11 updates the parameters of the personal model based on the observation data of the target subject and observation data limited to the range of use of another subject (step S8). Specifically, the parameters α_{A}^{X} and β_{A}^{Y} of the personal model for the testee X are updated based on the effect observation value R_{A}^{X} relating to the target subject and the effect observation value R_{A}^{Y} relating to another subject. The parameters of the personal model need not be updated based on both the observation data of the target subject and the observation data limited to the range of use of another subject, and the parameters of the personal model may be updated, for example, based only on the observation data limited to the range of use of another subject after the parameters of the personal model are updated based on the observation data of the target subject, or vice versa.

Thereby, the medical information processing according to the present embodiment ends.

Next, several embodiments according to the present embodiment will be described. In the working examples to be described below, it is assumed that the patient X is the target subject, and that the patient Y is another subject. It is also assumed that the personal model in the working examples to be described below is a model for calculating effect evaluation values of daily exercise amounts of a heart failure patient. Specifically, it is assumed that the personal model includes two types of structure models, namely, a structure model MX1 of calculating an effect evaluation value relating to a low-level exercise amount, and a structure model MX2 relating to a middle-level exercise amount. It is assumed that the structure model MX1 has a large effect expected value and a small variance value, compared to the structure model MX2. This means that the frequency with which a medical decision relating to a low-level exercise amount is assigned tends to be higher than the frequency with which a medical decision relating to a middle-level exercise amount is assigned. It is unlikely that a medical decision relating to a middle-level exercise amount is optimal, compared to a medical decision instructing a low-level exercise amount. It is normal for the patient X to hesitate to select a medical decision relating to a personal model for a middle-level exercise amount. However, if the patient X selects a medical decision relating to a middle-level exercise amount, it is possible to improve the estimation precision of the personal model for the patient X through effects etc. obtained by the medical decision. Such a selection of a medical decision that is not frequently selected and may cause damage in the short term but may help improve the estimation precision of the model is called an “exploration activity”. In the present case, selecting a medical decision relating to a middle-level exercise amount constitutes an exploration activity.

Assuming that the patient Y is capable of updating parameters of the personal model of the patient X based on observation data obtained by selecting a medical decision relating to a middle-level exercise amount, the incentive for the patient X to select a medical decision relating to a middle-level exercise amount will be reduced. On the other hand, if the observation data of a medical decision relating to a middle-level exercise amount obtained by a patient himself/herself by taking a risk is utilized by the patient X, the patient Y may have a feeling of unfairness. The processing circuitry 11 according to the present embodiment determines the range of use of the observation data of the patient Y to be used for updating the personal model of the patient X, to achieve incentive compatibility by allowing happiness of the individual patients to be consistent with happiness of all the patients.

### Working Example 1

The processing circuitry 11 according to Working Example 1 determines, with the determination function 114, a range of use of observation data of another subject based on a time of use of the personal model relating to the target subject.

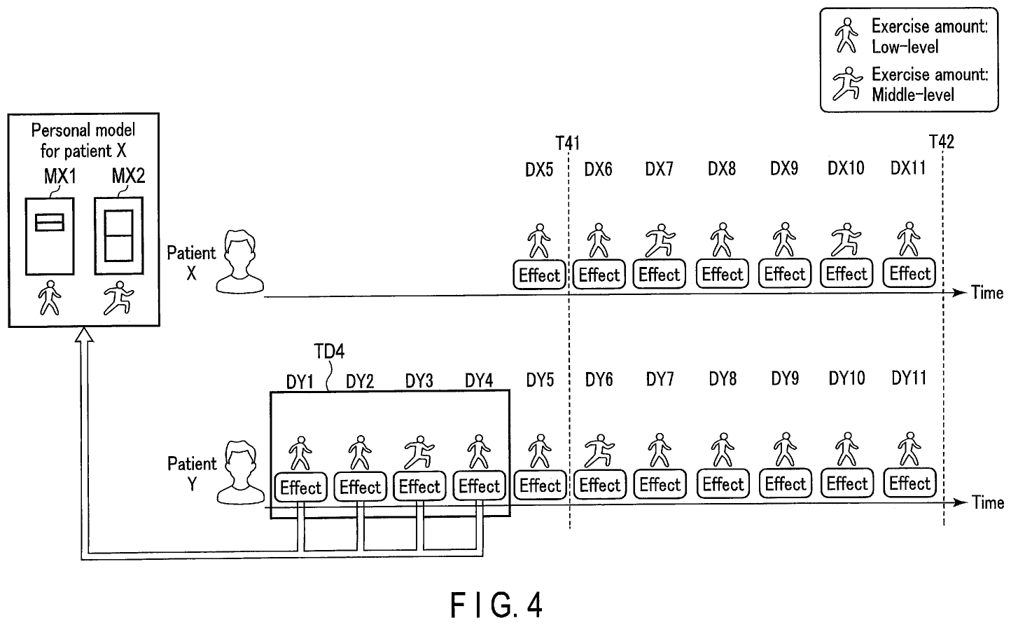

FIG. 4 is a diagram illustrating a range of use according to Working Example 1. In accordance with steps S1 to S4 in FIGS. 2 and 3, an assignment of a medical decision by the assignment function 111, observation of the effect by the observation function 112, and accumulation of the observation data by the accumulation function 113 are repeatedly performed for each of the patients X and Y.

It is assumed, as shown in FIG. 4, that the patient Y has been using a personal model (not illustrated) of the patient Y, etc. earlier than the patient X, and that observation data samples DY1-DY11 of the patient Y are accumulated in the database DB2. It is assumed, for example, that each of the observation data samples DY1, DY2, DY4, DY5, DY7, DYS, DY9, DY10, and DY11 includes an effect observation value produced as a result of performing an exercise of a low-level exercise amount. It is assumed that each of the observation data samples DY3 and DY6 includes an effect observation value produced as a result of performing an exercise of a middle-level exercise amount.

It is assumed, as shown in FIG. 4, that, for the patient X, an effect observation value has been observed for the first time at time T41. That is, time T41 is the time of start of use of the personal model of the patient X. It is assumed that, at time T42, the personal model for the patient X is determined to be updated at step S4. The processing circuitry 11 determines, with the determination function 114, a period TD4 prior to the start-of-use time T41 of the personal model relating to the patient X, which is the target subject, as a range of use of observation data of the patient Y who is another subject. The observation data of the patient Y during a period after the start-of-use time T41 is not included in the range of use.

Upon determining the range of use TD4, the processing circuitry 11 updates, with the updating function 116, the personal model of the patient X based on the observation data of the patient X and the observation data of the patient Y relating to the range of use TD4. Specifically, to update the structure model MX1 relating to a low-level exercise amount, observation data DX5, DX6, DX8, DX9, and DX11 of the patient X and observation data DY1, DV2, DY4, DY5, DY7, DY8, DY9, DY10, and DY11 of the patient Y relating to a low-level exercise amount are used. To update the structure model MX1 relating to a middle-level exercise amount, observation data DX7 and DX10 of the patient X and observation data DY3 and DY6 of the patient Y relating to a middle-level exercise amount are used.

In other words, the processing circuitry 11 detects, at time T41, that the patient X has taken an exercise of a low-level or middle-level exercise amount as an activity. The detection is performed by, for example, acquiring observation data corresponding to the activity, and accumulating the observation data in the database DB1. Triggered by the detection of the activity, the processing circuitry 11 may use observation data prior to time T41 of another patient Y. In other words, triggered by the detection of the activity, observation data after time T41 of another patient Y can no longer be used. That is, according to Working Example 1, the processing circuitry 11 updates the structure model MX based on the observation data after time T41 of the patient X and the observation data prior to time T41 of the patient Y.

In this manner, at a stage when the patient X considers making a medical decision using a personal model, parameters of the personal model can be updated by utilizing observation data of the patient Y limited to the observation data prior to the start-of-use time T41. Thereby, from the viewpoint of the patient X, since the structure model MX2 relating to a medical decision that is less likely to be optimal can be updated based on a large number of samples of observation data, without selecting the medical decision that is less likely to be optimal, the patient X will have a deep feeling of happiness. From the viewpoint of the patient Y, since the range of use is limited to the observation data prior to the start-of-use time T41, the feeling of unfairness is not high. It is thereby possible to achieve incentive compatibility by allowing the happiness of the individual patients to be consistent with the happiness of all the patients.

The range of use according to Working Example 1 need not necessarily be prior to the start of use of the personal model of the patient X who is the target subject. The processing circuitry 11 may, for example, determine a period of suspension of use of the personal model of the patient X as a range of use of the observation data of the patient Y. The period of suspension of use is a period during which the use of the personal model is suspended for some reason at or after the start-of-use time of the personal model. If the patient X has started using the personal model after the period of suspension of use has elapsed, the personal model of the patient X may be updated by utilizing the observation data of the patient Y accumulated in the period of suspension of use.

A case is assumed where the end-of-use time of the personal model is predetermined; for example, it could be a time immediately before the end of the treatment of the patient X. In this case, the processing circuitry 11 may determine, as the range of use of the observation data of the patient Y, a period going back in time from the end-of-use time of the personal model of the patient X by a statistically or dynamically determined period. As an example, the statistically determined period refers to a period determined in advance by the operator, and the dynamically determined period refers to a period determined in accordance with the contents of update of the individual model. During the period of use, the processing circuitry 11 updates the parameters of the personal model of the patient X based on the observation data of the patient X and the observation data of the patient Y accumulated during the period of use. The use of the observation data of the patient Y during such a period is permitted since the patient X is assumed to have a reduced incentive for performing an exploration activity, regardless of whether or not the observation data of the patient Y can be used immediately before the end of the personal model of the patient X.

Accordingly, the patient Y is not expected to have a great feeling of unfairness. On the other hand, the patient X can improve the precision of the personal model without performing an exploration activity by himself or herself. It is thereby possible to achieve incentive compatibility by allowing the happiness of the individual patients to be consistent with the happiness of all the patients.

### Working Example 2

The processing circuitry 11 according to Working Example 2 determines, with the determination function 114, a range of use of observation data of another subject based on a medical decision selected by the target subject. The description will use the same symbols for the structural components having substantially the same functions as those of Working Example 1, and repeated explanations will be given only if necessary.

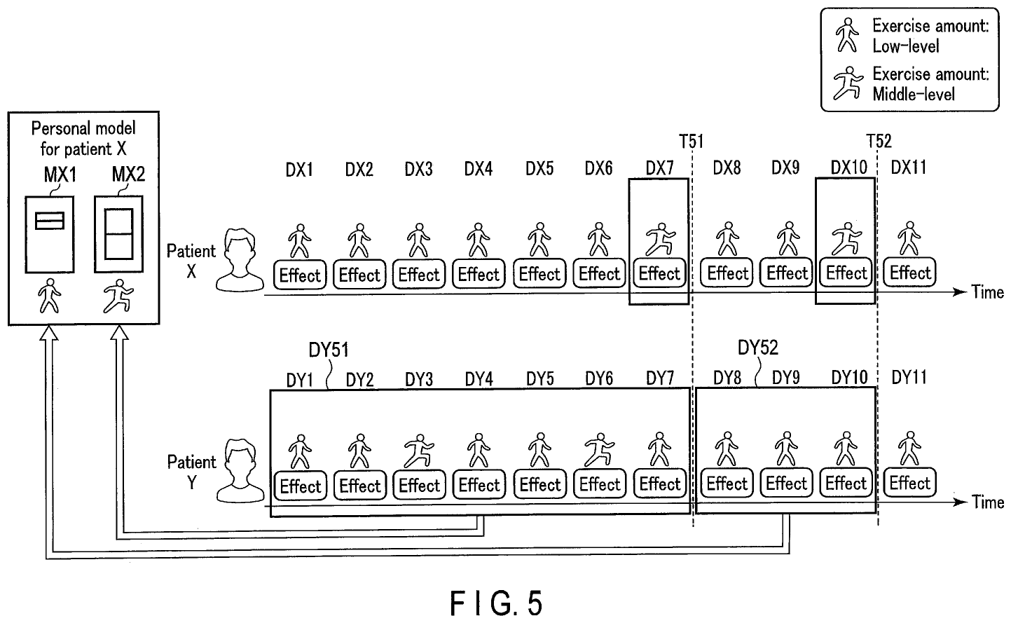

FIG. 5 is a diagram illustrating a range of use according to Working Example 2. As shown in FIG. 5, in Working Example 2, too, an assignment of a medical decision by the assignment function 111, observation of the effect by the observation function 112, and accumulation of the observation data by the accumulation function 113 are repeatedly performed for each of the patient X who is the target subject, and the patient Y who is another subject, in accordance with steps S1-S4 in FIGS. 2 and 3.

As shown in FIG. 5, if the patient X who is the target subject, has selected an exploration activity, the processing circuitry 11 according to Working Example 2 determines, with the determination function 114, a period prior to the processing time when the exploration activity has been selected as a range of use. The exploration activity is an activity corresponding to a medical decision determined to be less likely to be optimal by the personal model, or a medical decision other than a medical decision determined to be optimal by the personal model. The term “optimal” in the present embodiment means that the expected value of the effect evaluation value is maximum. It is to be noted, however, that the term “optimal” in the present embodiment is not limited to such a meaning, and may include the meaning that an index is most suitable for the target patient. Such an index may be of any type that satisfies this “optimal” condition.

It is assumed, for example, that in FIG. 5, performing an exercise of a middle-level exercise amount corresponds to an exploration activity, and that the patient X has selected an exploration activity at time T51. In this case, the processing circuitry 11 determines a period DY51 prior to time T51 as the range of use. The processing circuitry 11 updates parameters of the structure models MX1 and MX2 based on observation data DX7 of the patient X corresponding to time T51 and observation data DY1 to DY7 of the patient Y accumulated during the period DY51.

If the patient X has selected an exploration activity again at time T52, the processing circuitry 11 determines a period DY52 from time T51 when the patient X has selected the previous exploration activity to time T52 when the current exploration activity has been selected as the range of use. The processing circuitry 11 updates parameters of the structure models MX1 and MX2 based on observation data DX10 of the patient X corresponding to time T52 and observation data DY8 to DY10 of the patient Y accumulated during the period DY52.

In other words, the processing circuitry 11 detects that the patient X has performed an exploration activity at time T51 or T52. The detection is performed by, for example, acquiring observation data corresponding to the exploration activity, and accumulating the observation data in the database DB1. Triggered by the detection of the exploration activity, the processing circuitry 11 may use observation data prior to time T51 or T52 of another patient Y. The processing circuitry 11 updates the structure model MX based on the entirety of the observation data DX of the patient X and the observation data DY of the patient Y prior to the time of detection of the exploration activity.

In this manner, triggered by the selection of the exploration activity by the patient X, who is the target subject, the processing circuitry 11 according to Working Example 2 uses the observation data of the patient Y who is another subject, acquired prior to the time of selection of the exploration activity for updating the parameters of the personal model for the patient X. In other words, by allowing the observation data of the patient Y to be utilized through selection of the exploration activity, an incentive for selecting an exploration activity can be given to the patient X. The same applies to the patient Y who is another subject. Accordingly, according to Working Example 2, it is possible to achieve incentive compatibility by allowing the happiness of the individual patients to be consistent with the happiness of all the patients.

The processing circuitry 11 may determine a type of observation data of the patient Y during the range of use and/or a time width of the range of use, based on comparison between the effect evaluation value for the medical decision selected by the patient X who is the target subject, and the effect evaluation value for the medical decision selected by the personal model for the patient X. Specifically, the smaller the effect evaluation value for the medical decision selected by the patient X, compared to the effect evaluation value for the medical decision selected by the personal model, the greater the time width of the range of use settable by the processing circuitry 11. This is for the purpose of providing observation data pf a large volume to the patient X as a reward, since the patient X is considered to have selected an exploration activity with a large risk.

As another example, the smaller the effect evaluation value for the medical decision selected by the patient X, compared to the effect evaluation value for the medical decision selected by the personal model, the greater the number of types of observation data of the patient Y included in the range of use used for updating that can be handled by the processing circuitry 11. If, for example, an exercise of a middle-level exercise amount has been selected as an exploration activity, the processing circuitry 11 uses both the observation data relating to an exercise of a middle-level exercise amount and observation data relating to an exercise of a low-level exercise amount for updating. If, on the other hand, an exercise of a low-level exercise amount, which is not an exploration activity, has been selected, the processing circuitry 11 uses only the observation data relating to an exercise of a low-level exercise amount for updating. In this manner, by appropriately controlling the type of the observation data and/or the time width of the range of use, an appropriate incentive can be given to the patient X.

### Working Example 3

The processing circuitry 11 according to Working Example 3 determines, with the determination function 114, the range of use of the observation data of another subject based on a similarity between the feature amount of the target subject and the feature amount of another subject. The description will use the same symbols for the structural components having substantially the same functions as those of Working Example 1, and repeated explanations will be given only if necessary.

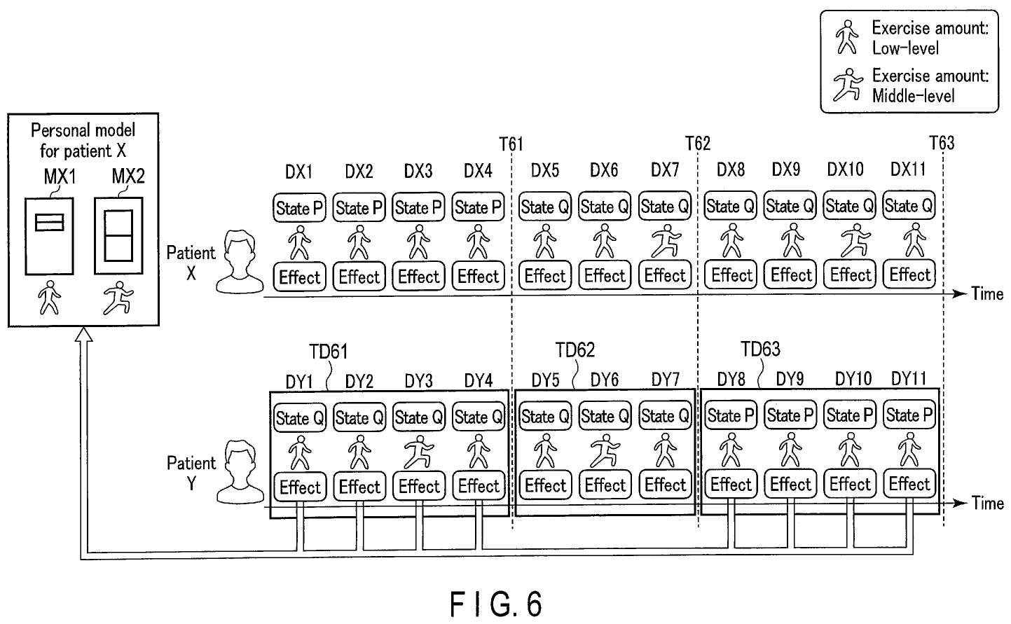

FIG. 6 is a diagram illustrating a range of use according to Working Example 3. As shown in FIG. 6, in Working Example 3, too, an assignment of a medical decision by the assignment function 111, observation of the effect by the observation function 112, and accumulation of the observation data by the accumulation function 113 are repeatedly performed for each of the patient X who is the target subject, and the patient Y who is another subject, in accordance with steps S1 to S4 in FIGS. 2 and 3.

It is assumed that the personal model according to Working Example 3 follows a contextual bandit algorithm, in which an effect evaluation value is calculated by taking the feature amount of the patient into consideration. As described above, the feature amount includes attributes that are information items which, by nature, are not changed by a medical decision just performed, and they include the sex, age, etc. of a testee, and/or conditions that are information items which, by nature, are changed according to a medical decision just performed, and they include a current blood pressure, a blood pressure value, a current blood glucose level, etc. of a testee. The processing circuitry 11 allows another subject for which a feature amount with a low similarity has been observed to be included in the range of use, and allows another subject for which a feature amount with a high similarity has been observed to be excluded from the range of use. Furthermore, the processing circuitry 11 allows a period during which a feature amount with a low similarity has been observed to be included in the range of use, with respect to the subject for which a feature amount with a low similarity has been observed. In this manner, the processing circuitry 11 according to Working Example 3 controls the human range as well as a temporal range as the range of use.

It is assumed that, as an example, the feature amount according to FIG. 6 takes two values, namely, a state P or a state Q. It follows that each sample of the observation data DX1 to DX11 and DY1 to DY11 includes an effect observation value and a state P or a state Q. In the observation data DX1 to DX4 and DY1 to DY4 prior to time T61, the state of the patient X is the state P, and the state of the patient Y is the state Q, with the similarity between the state of the patient X and the state of the patient Y being “0”. In the observation data DX5 to DX7 and DY5 to DY7 during a period from time T61 to time T62, the states of both the patient X and the patient Y are the state Q, with the similarity being “1”. In the observation data DX8 to DX11 and DY8 to DY11 during a period from time T62 to time T63, the state of the patient X is the state Q, and the state of the patient Y is the state P, with the similarity being “0”.

At the processing time T63, the processing circuitry 11 determines, with the determination function 114, the range of use. Specifically, the processing circuitry 11 compares, for each sample of observation data, the state of the patient X and the state of another subject such as the patient Y and a patient Z (not illustrated) accumulated prior to the processing time T63, and explores another subject having a state different from the state of the patient X. If another subject having a state different from the state of the patient X exists, the processing circuitry 11 allows said another subject to be included in the range of use, and allows another subject having a state identical to the state of the patient X to be excluded from the range of use. If multiple other subjects having a state different from the state of the patient X exist, all of them may be included in the range of use, or one of them may be included in the range of use. In FIG. 6, it is assumed that, since the state of the patient Y during the period TD61 prior to time T61 and the state of the patient Y during the period TD3 from time T62 to time T63 are different from those of the patient X, the patient Y is included in the range of use. Subsequently, the processing circuitry 11 allows the observation data DY1 to DY4 and DY8 to DY11 during the periods TD61 and TD63, during which a feature amount with a low similarity has been observed to be included in the range of use. The processing circuitry 11 updates parameters of the personal model for the patient X based on the observation data DY1 to DY4 and DY8 to DY11 during the period TD61 and the period TD63 of the patient Y.

In other words, the processing circuitry 11 decides, at each time, whether or not the feature amount of the patient X differs from the feature amount of the patient Y, and detects that the feature amount of the patient X is different from the feature amount of the patient Y. The detection is performed by, for example, acquiring observation data corresponding to the feature amount, and accumulating the observation data in the database DB1. The processing circuitry 11 can use observation data of the patient Y corresponding to the feature amount different from the patient X. The processing circuitry 11 updates the structure model MX based on the entirety of the observation data DX of the patient X and the observation data DY of the patient Y with a feature amount different from that of the patient X.

If, for example, the state is sex, the observation data of a patient with an identical sex will not be used for updating the personal model, but the observation data of a patient with a different sex will be used for updating the personal model. As another example, if the state is age, observation data of a patient who is close in age will not be used for updating the personal model, but observation data of a patient who greatly differs in age will be used for updating the personal model. This is because updating the personal model of the patient X by utilizing the observation data of a patient with a feature amount greatly different from that of the patient X causes a low effect on a medical decision performed by the patient X, thus not reducing an incentive for the patient X to select an exploration activity.

If attention is focused on a variable state, the human range of the range of use may be suitably changed at a timing when the state of each patient has changed. If, for example, the blood pressure of the patient X is low, the processing circuitry 11 updates the personal model by utilizing the observation data of the patient Y with a high blood pressure, but does not update the personal model by utilizing the observation data of the patient Z with a low blood pressure. At a timing when the blood pressure of the patient X has rapidly increased, the processing circuitry 11 may update the model by utilizing the observation data of the patient Z with a low blood pressure. As another example, if the blood pressure of the patient X is low, the processing circuitry 11 updates the personal model by utilizing the observation data of the patient Y with a high blood pressure, and does not update the personal model by utilizing the observation data of the patient Z with a low blood pressure; however, if the blood pressure of the patient Y has decreased and the blood pressure of the patient Z has increased thereafter, the model may be updated by utilizing the observation data of the patient Y with a low blood pressure. In this manner, by adaptively changing the human range according to the state of each patient, it is possible to maintain an incentive for the patient X to select an exploration activity without depending on variations in the states of the patient X and other subjects.

This completes the description of the working examples.

### Others

Various modifications can be made to the present embodiment. In the above-described embodiment, the assignment function 111, the observation function 112, the accumulation function 113, the determination function 114, the acquisition function 115, the updating function 116, and the display control function 117 are installed in a single computer. However, the present embodiment is not limited thereto. These functions 111 to 117 may be installed in multiple computers in a distributed manner. In other words, the medical information processing apparatus 1 may be a computer system configured by multiple computers in which the functions 111 to 117 are installed in a distributed manner. In addition, it is assumed that the databases DB1 and DB2 in which the observation data is accumulated are external computers of the medical information processing apparatus 1. However, the present embodiment is not limited thereto, and the databases DB1 and DB2 may be included in the medical information processing apparatus 1.

As another example, with the determination function 114, the processing circuitry 11 may determine, according to the state of the personal model relating to the target subject, the range of use of the observation data of another subject to be passed for updating the personal model. The state of the personal model is specifically expressed by the number of samples of observation data used for training each structure model, property index values, etc. of each structure model. The property index values are parameters of a mathematical expression of each structure model, and the parameters α, β, p, etc. can be used, for example.

A case will be described, as an example, where the state of the personal model is the number of samples of observation data used for training each structure model (hereinafter referred to as “training data”). When the number of samples of training data is small, selecting a medical decision corresponding to the structure model refers to performing an exploration activity. The processing circuitry 11 compares, for example, the number of samples of training data of each structure model with a threshold value. It is assumed that the number of samples of training data of the structure model MX1 is larger than a threshold value, and that the number of samples of training data of the structure models MX2 is smaller than the threshold value. If the number of samples of training data is larger than the threshold value, the processing circuitry 11 determines, as a range of use, observation data of another subject Y relating to a medical decision corresponding to the structure model MX1, and trains the structure model MX1 using observation data of the subject Y included in the determined range of use. If the number of samples of training data is smaller than the threshold value, the processing circuitry 11 does not determine, as a range of use, the observation data of another subject Y relating to a medical decision corresponding to the structure model MX2, and does not use the observation data of the subject Y for training the structure model MX2. It is to be noted that, to determine which of the numbers of samples of training data is larger/smaller, the number of samples of training data of a structure model may be compared with that of another structure model, instead of comparing the number of samples of training data with a threshold value.

### Remarks

According to some of the above-described working examples, the medical information processing apparatus 1 according to the present embodiment includes processing circuitry 11. With the updating function 116, the processing circuitry 11 updates a personal model relating to the target subject and adapted to calculate an effect evaluation value for a medical decision based on observation data corresponding to the effect of the medical decision on the target subject and observation data limited to a partial range of observation data corresponding to the effect of the medical decision on another subject different from the target subject.

With the above-described configuration, since the observation data of another subject utilized for updating the personal model of the target subject is limited to the partial range (range of use), it is possible to increase the feeling of happiness of the target subject while suppressing the feeling of unfairness of another subject in updating the personal model of the target subject. It is thereby possible to achieve incentive compatibility by allowing the happiness of the individual patients to be consistent with the happiness of all the patients. In accordance therewith, it is possible to improve the precision of the personal model, thereby realizing personalization of medicine (precision medicine). Through realization of the personalization of medicine, it is possible to construct a model based on a small amount of observation data, compared to a general-purpose model. Since elimination of the individual characteristics need not be taken into consideration, it can be expected that a model can be constructed without conducting a randomized controlled trial.

According to at least one of the embodiments described above, it is possible to achieve incentive compatibility in assigning medical decisions.

The term “processor” used herein refers to, for example, a CPU or a GPU, or various types of circuitry, such as an application-specific integrated circuit (ASIC), a programmable logic device (e.g., a simple programmable logic device (SPLD), a complex programmable logic device (CPLD), or a field programmable gate array (FPGA)), and so on. The processor reads programs stored in storage circuitry and executes them to realize the intended functions. The programs may be incorporated directly in circuits of the processor, instead of being stored in the storage circuitry. According to such architecture, the processor reads the programs incorporated in its circuits and executes them to realize the functions. As another option, functions corresponding to the programs may be realized by a combination of logic circuits, instead of having the programs executed. The embodiments, etc., described herein do not limit each processor to a single circuitry-type processor. Multiple independent circuits may be combined and integrated as one processor to realize the intended functions. Furthermore, multiple components or features as given in FIG. 1 may be integrated as one processor to realize the respective functions.

While certain embodiments have been described, they have been presented by way of example only, and are not intended to limit the scope of the inventions. Indeed, the novel embodiments described herein may be embodied in a variety of other forms. Furthermore, various omissions, substitutions, and changes in the form of the embodiments may be made without departing from the spirit of the inventions. The accompanying claims and their equivalents are intended to cover such forms or modifications as would fall within the scope and spirit of the inventions.

What is claimed is:

1. A medical information processing apparatus comprising:
processing circuitry which updates a personal model relating to a target subject, the personal model being calculating an effect evaluation value for a medical decision on the target subject, the processing circuitry updating the personal model based on first data corresponding to an effect of the medical decision on the target subject and data limited to a partial range of second data corresponding to an effect of a medical decision on another subject different from the target subject.

2. The medical information processing apparatus according to claim 1, wherein
the processing circuitry determines the partial range based on a period of use of the personal model relating to the target subject.

3. The medical information processing apparatus according to claim 2, wherein
the processing circuitry determines, as the partial range, a period prior to a time of start of use of the personal model, a period of suspension of use of the personal model, or a period going back in time from a time of end of use of the personal model by a statistically or dynamically determined period.

4. The medical information processing apparatus according to claim 1, wherein
the processing circuitry determines the partial range based on a medical decision selected by the target subject.

5. The medical information processing apparatus according to claim 4, wherein
the processing circuitry determines, if the target subject has selected, at a target time, a medical decision determined to be less likely to be optimal by the personal model or a medical decision other than a medical decision determined to be optimal by the personal model, a period prior to the target time as the partial range.

6. The medical information processing apparatus according to claim 5, wherein
the processing circuitry determines, based on comparison between an effect evaluation value for the medical decision selected by the target subject and an effect evaluation value for the medical decision selected by the personal model, a type of the partial range of the second data used for updating the personal model and/or a time width of the partial range.

7. The medical information processing apparatus according to claim 1, wherein
the processing circuitry determines the partial range based on a similarity between a feature amount of the target subject and a feature amount of said another subject.

8. The medical information processing apparatus according to claim 7, wherein
the processing circuitry allows another subject for which a feature amount with a low similarity has been observed to be included in the partial range, and allows another subject for which a feature amount with a high similarity has been observed to be excluded from the partial range.

9. The medical information processing apparatus according to claim 8, wherein
the processing circuitry determines a period during which the feature amount with the low similarity has been observed as the partial range, among the observation period for the second data of the another subject.

10. The medical information processing apparatus according to claim 1, wherein
the processing circuitry is configured to:
acquire the second data from a database; and
extract the data limited to the partial range from the second data.

11. The medical information processing apparatus according to claim 1, wherein
the processing circuitry acquires the data limited to the partial range of the second data from a database.

12. The medical information processing apparatus according to claim 1, wherein
the first data is series data including multiple effect observation values respectively corresponding to multiple observation times of the effect of the medical decision on the target subject, and
the second data is series data including multiple effect observation values respectively corresponding to multiple observation times of the effect of the medical decision on said another subject.

13. The medical information processing apparatus according to claim 1, wherein
the target subject and said another subject each comprise an individual testee, a group of testees including at least one testee, a hospital, or a hospital group including at least one hospital.

14. The medical information processing apparatus according to claim 1, wherein
the processing circuitry updates, based on the first data and the partial range of the second data determined based on an activity and/or a feature amount of the target subject.

15. The medical information processing apparatus according to claim 1, wherein
the processing circuitry determines the partial range of the second data to be passed for updating the personal model according to a state of the personal model relating to the target subject.

16. A medical information processing method comprising:
acquiring first data corresponding to an effect of a medical decision on a target subject;
acquiring a data limited to a partial range of a second data corresponding to an effect of a medical decision on another subject different from the target subject; and
updating a personal model relating to the target subject, based on the first data and the data limited to the partial range, a personal model being calculating an effect evaluation value for the medical decision.

17. A non-transitory computer-readable recording medium storing a medical information processing program which causes a circuitry to perform functions:
acquiring first data corresponding to an effect of a medical decision on a target subject;
acquiring a data limited to a partial range of a second data corresponding to an effect of a medical decision on another subject different from the target subject; and
updating a personal model relating to the target subject, based on the first data and the data limited to the partial range, a personal model being calculating an effect evaluation value for the medical decision.
